(19) United States
(12) Patent Application Publication
(10) Pub. No.: US 2011/0189649 A1
(43) Pub. Date: Aug. 4, 2011

(54) Laboratory Apparatus for a Controlled Environment

(76) Inventor: Andrew Skinn, West Yorkshire (GB)

(21) Appl. No.: 13/003,340

(22) PCT Filed: Jul. 8, 2009
(86) PCT No.: PCT/GB2009/001699

§ 371 Date: Apr. 22, 2011

(30) Foreign Application Priority Data
Jul. 8, 2008 (GB) 0812477.8

(51) Int. Cl. A01N 1/02; C12M 1/00

(52) U.S. Cl. 435/2; 435/289.1

(57) ABSTRACT

A controlled environment system for use in IVF, ICSI or other biological procedures has a main chamber containing one or more manipulator devices and a microscopy viewing system enabling viewing of the manipulation procedure. The proportions of gas in a multi gas chamber atmosphere set up in the main chamber can be varied and the gas atmosphere in the main chamber can be heated. A recirculation gas circuit enables recirculation of the chamber gas atmosphere, the recirculation gas circuit including a particulate filter arrangement. The chamber provides a sealed atmosphere for conducting the manipulation procedure remotely from the exterior of the chamber in an environment in which the atmosphere is sealed from the external ambient atmosphere and the chamber atmosphere is re-circulated and filtered.

### BACKGROUND OF THE INVENTION

1. Field of the Invention

The present invention relates a laboratory apparatus particularly suited for use in, but not limited for use in, In-Vitro Fertilisation (IVF).

2. State of the Art

IVF treatment has gained popularity over the last twenty five years providing the opportunity for infertile couples to improve chances of reproduction. The female is treated with hormones such that a large number of unfertilised eggs can be extracted and taken to a laboratory where they are treated under carefully controlled conditions.

The unfertilised eggs are washed, sorted and fertilised and then transferred to an incubating environment which is arranged to as closely as possible resemble the conditions inside the body. For this reason, careful control is made of the temperature, humidity and gas concentrations Once the fertilised eggs have reached embryonic stage, the best embryos are selected and implanted into the female.

As the conditions under which the embryo forms are critical and must closely resemble those found in the body, an apparatus must be provided that achieves and constantly maintains such a suitable environment in terms of temperature, humidity and concentration of mixed gasses. It is known that a high proportion of IVF failures are due to the effects of the environment, which is typically controlled in an active chamber. An example of such a chamber is disclosed in WO-A-2005/040330.

In so called Intracytoplasmic Sperm Injection (ICSI) techniques, a single sperm is injected directly into a respective egg. The procedure is typically carried out under a microscope using multiple micromanipulation devices (micromanipulators, microinjectors and micropipettes). A holding pipette stabilizes the selected oocyte and a needle is pierced through the oolemma and into the inner part of the oocyte. The distal end of the needle is then loaded with a single selected sperm and released into the oocyte.

Prior art controlled atmosphere IVF chambers an apparatus have not been suitable for use in certain IVF procedures, such as ICSI.

An improved apparatus for use in IVF procedures has now been devised.

According to a first aspect, the present invention provides a controlled environment system for use in IVF or other biological procedures, the system comprising:
a main chamber containing:
   one or more manipulator devices for manipulating items in the interior of the chamber, and
   a microscopy viewing system enabling viewing of the manipulation procedure;
means for modifying the proportions of gas in a multi gas chamber atmosphere set up in the main chamber;
chamber heater means for heating the gas atmosphere in the main chamber;
a recirculation gas circuit providing for recirculation of the main chamber gas atmosphere, the recirculation gas circuit including a particulate filter arrangement.

According to a second aspect, the present invention provides method of conducting a biological procedure in a controlled environment in which an ICSI or another procedure for sperm injection is conducted on an oocyte in a controlled atmosphere of a chamber and subsequent incubation is carried out in the same chamber, the environment being controlled to have a predetermined gas mix atmosphere, a predetermined chamber temperature and the atmosphere being re-circulated via a recirculation loop extending outside the chamber which includes a filter function.

The chamber provides a sealed atmosphere for conducting the manipulation procedure remotely from the exterior of the chamber in an environment in which the atmosphere is sealed from the external ambient atmosphere and the chamber atmosphere is re-circulated and filtered. Recirculation and filtration is important for the air sealed in the chamber to ensure removal of contaminants such as volatile organic compounds (VOCs) from the controlled atmosphere, that may have been introduced into the chamber with the organic matter. This is particularly the case because incubation may continue in the sealed chamber for extended periods of time. Recirculation of the chamber atmosphere also induces an airflow in the chamber which is beneficial for the incubation and IVF procedures.

It is preferred that the system includes recirculation gas circuit heater means for heating the gas in the recirculation gas circuit. This enables condensation within the recirculation circuit to be avoided, which is important to maintain a consistent atmosphere in the main chamber.

It is preferred that the method according to the invention is carried out using the system of the present invention.

It is envisaged that the recirculation gas heater could also act as the heater for the main chamber atmosphere, however in certain embodiments a separate main chamber heater will be utilised.

The chamber beneficially has a sealed entry port for the passage of items into and out of the main chamber. The system preferably includes an interlock chamber for the passage of items into and out of the main chamber. The interlock chamber has a first interlock door communication with the external environment, and a second interlock door communicating with the chamber.

Beneficially, the system includes an air agitation system for the main chamber to enable circulation of air within in the main chamber itself.

The system is beneficially provided with remote operation means enabling remote operation of the manipulation device from externally of the chamber. In one embodiment the chamber is provided with glove or gauntlet barrier ports, enabling the user to have hand access into the interior of the main chamber with a hand received in the barrier glove or gauntlet communicating via a wall of the main chamber. Other remote operation means may be provided. Particularly for ICSI procedures it is preferred that remote operation means is provided permitting operation of the ICSI injector device from externally of the chamber.

For ICSI procedures it is preferred that the chamber includes an ICSI workstation including one or more ICS injection devices remotely operable and targeted upon a working zone which is also targeted by a microscopy viewing arrangement which includes an optical display viewable from externally of the chamber.

In an embodiment particularly suited to the system of the present invention, the one or more manipulator devices may comprise an ICSI micro-injection device and or other ICSI devices.

Beneficially the main chamber is provided with a humidification system for modifying the humidity of the main chamber atmosphere.

The recirculation gas circuit has a circuit path via the filter and the sterilisation arrangement which is outside the main chamber.

The filter arrangement may comprise a medical application HEPA filter.

The sterilization arrangement may comprise a UV sterilisation device.

The sterilisation filter is provided flow-wise upstream of the filter arrangement in the gas recirculation circuit. This ensures that any microorganisms are killed before they enter the filter.

The invention will now be further described in a specific embodiment, by way of example only, and with reference to the accompanying drawings.

### BRIEF DESCRIPTION OF THE DRAWINGS

FIG. 1 is a schematic representation of a controlled environment system in accordance with the present invention; and

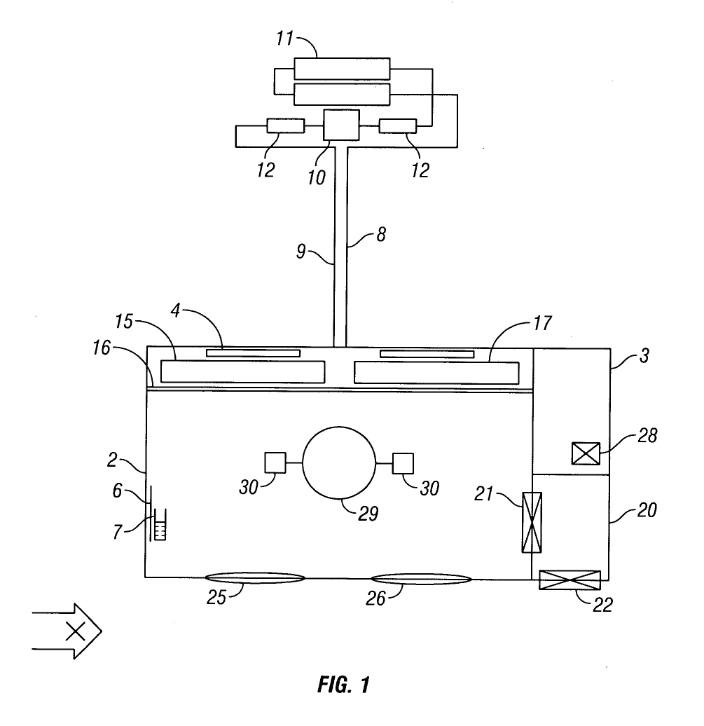

FIG. 2 is a side view of a part of the system of FIG. 1.

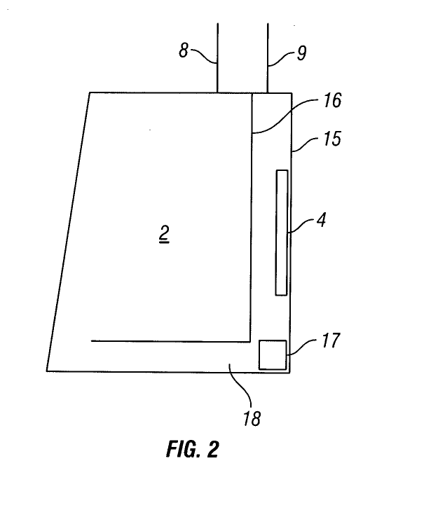

### DETAILED DESCRIPTION OF THE PREFERRED EMBODIMENTS

Referring to the drawings, there is shown a controlled environment apparatus 1 comprising a primary working chamber 2 for providing a controlled atmosphere for manipulation and/or culture of living biological material such as sperm, oocytes, or fertilised zygotes or embryos. The gas concentration proportion between oxygen, nitrogen and carbon dioxide is carefully monitored and controlled by a gas supply and mixer unit 3. For example the controller (not shown) such a programmable logic controller (PLC) may be set to maintain the chamber atmosphere gas mix at required predetermined concentration levels. A user interface device such as a user interactive control panel is typically provided to enable parameters to be manually reconfigured.

The chamber environment gas temperature is controlled by the PLC controller, in combination with a plate heater 4 mounted in a plenum 15 behind the rear wall 16 of the chamber. A duct 18, is provided at the bottom of the rear chamber wall 16 which in combination with a circulation fan 17 acts to draw gas into the main chamber 2, along the plenum 15 and past the heater to heat the air. Operation of the heater 4 and the circulation fan is controlled by the PLC controller.

Humidity in the chamber is also controlled by the PLC which controls operation of a humidifier 6 provided adjacent a water trough 7. Operation of the heater increases the humidity in chamber 2.

In this manner a controlled environment in terms of temperature, humidity and gas atmosphere mix can be maintained in the chamber 2. However for procedures in which fertilization and incubation are carried out in the same chamber, for example ICSI procedures envisaged to be carried out in the apparatus of the present invention, it is believed to be important to ensure that the air is filtered and recirculated (with appropriate care being taken to ensure that other characteristics of the atmosphere of the chamber 2 are maintained).

In accordance with the system of the present invention, the chamber atmosphere is drawn into a ‘closed loop’ external recirculation circuit which leaves the chamber via sealed outlet 8 and is returned via sealed inlet 9. The recirculation loop is provided with a fan 10 to draw out the chamber atmosphere into the recirculation loop and direct it to an in-line ultraviolet light sterilization unit 11 and subsequently a HEPA filtration system 12 comprising a pair of HEPA filters arranged in series. This arrangement can be used to sterilise and to filter out particulates present (including bacteria and viruses). The light sterilization unit 11, where present, can be positioned flow-wise upstream of the HEPA filtration system 12 in order to ensure that bacteria/viruses etc are killed of before entering the filtration system 12. It has however been found that appropriate filter devices can perform adequately without additional sterilisation being required. A separate sterilisation device, in addition to the filter device is therefore an optional extra for certain operable systems.

The UV and HEPA units can be replaced off-line without the need to enter the main chamber. The system ensures that the air re-circulated to the main chamber 2 is in accordance with the requirements of ‘Class B’.

Recirculation and filtration is important for the air sealed in the chamber 2 to ensure removal of contaminants such as volatile organic compounds (VOCs) from the controlled atmosphere, that may have been introduced into the chamber with the organic matter. This is particularly the case because incubation may continue in the sealed chamber for extended periods of time. Recirculation of the chamber atmosphere also induces an airflow in the chamber which is beneficial for the incubation and IVF procedures.

The system is provided with an interlock chamber 20 having a sealed interlock port 21 in communication with the main chamber 2. A sealed interlock port 22 provides access into the chamber. The interlock chamber 20 may be arranged to evacuate environmental air and filled with the atmosphere of the chamber. An evacuation pump 28 is provided for this purpose. The interlocks 21, 22 can be set at a gas bleed setting to allow the interlock chamber 20 to fill with ambient air from outside the system, or the atmosphere of the chamber 2. In a preferred realisation, when interlock door 22 is opened, ambient air will enter the interlock when the interlock door 22 is closed a pump is operated to vent atmosphere from the chamber 2 and exhaust the contents of interlock camber 20 to atmosphere. The interlocks 21, 20 operated in sequence will enable the impact of external ambient conditions to have minimal impact on the atmosphere of the chamber when objects are brought into the main chamber 2 via the interlock 20.

An inspection microscope 29 is positioned internally of the main chamber 2, connected to a visual display screen which may be positioned outside the main chamber or more beneficially from a user perspective, internally of the chamber. A manipulator device such as an ICSI micro-injector device 30 is positioned in the interior of the main chamber 2. The microscopy system enables the user to operate the micro manipulator device 30 in an accurate and controlled manner. For ICSI and other IVF systems, a workstation is effectively provided in the controlled and sealed atmosphere re-circulated in the chamber including one or more ICS injection or other devices remotely operable and targeted upon a working zone which is also targeted by a microscopy viewing arrangement which includes an optical display viewable from externally of the chamber.

The front wall of the main chamber is typically transparent and provided with a pair of gloved ports 25 26, into which a person can insert their hands and arms in order to facilitate manipulation of the micro devices (such as micro-injection device 30) and other items within the interior of the main chamber 2 whilst still providing a sealed barrier between the interior of the chamber and the chamber exterior environment.

In use for the ICSI process, the introduction of the sperm into the oocyte can take place in the controlled environment of the main chamber 1 in which the atmosphere is continuously controlled in terms of temperature, humidity and chamber gas atmosphere mix. Also the atmosphere is re-circulated via a filter (and optionally sterilisation) route to meet predetermined standards (for example ‘Class B’ air standard), remove VOCs and provide an airflow in the chamber. The microscope and micromanipulators present in the chamber 2 are used for carrying out the procedure, but are operated from remotely of the chamber. Following cytoplasm injection the zygotes can be maintained in the environment of the main chamber and development monitored. There is thus no need to have sperm injection and subsequent incubation carried out in separate environments or atmospheres. This enhances the chances of viable embryos forming.

1. A controlled environment system for use in a biological procedure, the system comprising:
a main chamber containing:
one or more manipulator devices for manipulating items in the interior of the chamber, and
a microscopy viewing system enabling viewing of the manipulation procedure;
means for modifying the proportions of gas in a multi gas chamber atmosphere set up in the main chamber;
chamber heater means for heating the gas atmosphere in the main chamber; and
a recirculation gas circuit providing for recirculation of the main chamber gas atmosphere, the recirculation gas circuit including a particulate filter arrangement.

2. A system according to claim 1, wherein:
the chamber provides a sealed atmosphere for conducting the manipulation procedure remotely from the exterior of the chamber in an environment in which the atmosphere is sealed from the external ambient atmosphere and the chamber atmosphere is re-circulated and filtered.

3. A system according to claim 1, wherein:
the recirculation gas circuit includes a sterilization arrangement.

4. A system according to claim 1, further comprising:
recirculation gas circuit heater means for heating the gas in the recirculation gas circuit.

5. A system according to claim 1, wherein:
the chamber has a sealed entry port for the passage of items into and out of the main chamber.

6. A system according to claim 1, further comprising:
an interlock chamber for the passage of items into and out of the main chamber.

7. A system according to claim 1, further comprising:
an air agitation system for the chamber to enable circulation of air within in the main chamber itself.

8. A system according to claim 1, further comprising:
remote operation means enabling remote operation of the manipulation device from externally of the chamber.

9. A system according to claim 8, wherein:
the chamber is provided with glove or gauntlet barrier ports, enabling the user to have hand access into the interior of the main chamber with a hand received in the barrier glove or gauntlet communicating via a wall of the main chamber.

10. A system according to claim 1, wherein:
the one or more manipulator devices comprise an ICSI micro-injection device.

11. A system according to claim 1, wherein:
the main chamber is provided with a humidification system for modifying the humidity of the main chamber atmosphere.

12. A system according to claim 1, wherein:
the recirculation gas circuit has a circuit path via the particulate filter arrangement which is outside the main chamber.

13. A system according to claim 1, wherein:
the particulate filter arrangement comprises a HEPA filter.

14. A system according to claim 1, wherein:
the recirculation gas circuit includes a sterilization arrangement, the sterilization arrangement comprising a UV sterilization device.

15. A system according to claim 1, further comprising:
a sterilization device disposed flow-wise upstream of the particulate filter arrangement in the gas recirculation circuit.

16. A system according to claim 1, further comprising:
a workstation including one or more devices remotely operable and targeted upon a working zone which is also targeted by a microscopy viewing arrangement which includes an optical display viewable from externally of the chamber.

17. A method of conducting a biological procedure in a controlled environment in which sperm injection is conducted on an oocyte in a controlled atmosphere of a chamber and subsequent incubation is carried out in the same chamber, the environment being controlled to have a predetermined gas mix atmosphere, a predetermined chamber temperature and the atmosphere being re-circulated via a recirculation loop extending outside the chamber which includes a filter device.

18. A method according to claim 17, wherein:
the chamber provides a sealed atmosphere for conducting the manipulation procedure remotely from the exterior of the chamber in an environment in which the atmosphere is sealed from the external ambient atmosphere and the chamber atmosphere is re-circulated and filtered.

19. A controlled environment system for use in intracytoplasmic sperm injection (ICSI) procedures, the system comprising:
a main chamber containing:
an ICSI injector device, and
a microscopy viewing system enabling viewing of the ICSI injection procedure;
remote operation means permitting operation of the ICSI injector device from externally of the chamber;
means for modifying the proportions of gas in a multi gas chamber atmosphere set up in the main chamber;
heater means for heating the gas atmosphere in the main chamber; and
a recirculation gas circuit providing for recirculation of the main chamber gas atmosphere, the recirculation gas circuit including a particulate filter arrangement.

20. A system according to claim 19, wherein:
the chamber provides a sealed atmosphere for conducting the ICSI procedure remotely from the exterior of the chamber in an environment in which the atmosphere is sealed from the external ambient atmosphere and the chamber atmosphere is re-circulated and filtered.
